(19) United States
(12) Patent Application Publication
(10) Pub. No.: US 2014/0303199 A1
(43) Pub. Date: Oct. 9, 2014

(54) INHIBITORS OF RGS PROTEINS

(71) Applicant: University of Iowa Research Foundation, Iowa City, IA (US)

(72) Inventor: David Roman, Iowa City, IA (US)

(73) Assignee: University of Iowa Research Foundation, Iowa City, IA (US)

(21) Appl. No.: 14/245,718

(22) Filed: Apr. 4, 2014

Related U.S. Application Data
(60) Provisional application No. 61/809,242, filed on Apr. 5, 2013.
(60) Provisional application No. 61/813,059, filed on Apr. 17, 2013.

(51) Int. Cl. A61K 31/56; A61K 31/352; A61K 31/4355

(52) U.S. Cl. CPC A61K 31/56; A61K 31/4355; A61K 31/352
USPC 514/279; 514/557; 514/453

(57) ABSTRACT

G-protein coupled receptors are a diverse group that are the target of over 50% of marketed drugs. Activation of these receptors results in the exchange of bound GDP for GTP in the Gα subunit of the heterotrimeric G-protein. The Gα subunit dissociates from the β/γ subunits and both proceed to affect downstream signaling targets. The signal terminates by the hydrolysis of GTP to GDP and is regulated by Regulators of G-protein Signaling (RGS) proteins that act as GTPase Activating Proteins (GAPs). This makes RGS proteins potentially desirable targets for “tuning” the effects of current therapies as well as developing novel pharmacotherapies. The present invention provides a convenient assay for identifying modulators of RGS proteins that can be adapted for high throughput screening utilizing the colorimetric dye, malachite green, to detect free phosphate in the presence of a test compound.

### RELATED APPLICATIONS

This application claims priority to U.S. Provisional Patent Application No. 61/809,242, filed Apr. 5, 2013, and to U.S. Provisional Patent Application No. 61/813,059, filed Apr. 17, 2013, the entirety of which are incorporated herein by reference.

### FIELD OF THE INVENTION

The present invention relates to inhibitors of regulators of G-protein signaling and methods of screening for such inhibitors. In particular, the present invention provides inhibitors of regulator of G-protein signaling proteins and methods of using such inhibitors to modulate physiological effects of G protein and receptor signaling.

### BACKGROUND OF THE INVENTION

G-protein coupled receptors (GPCRs) are a diverse group of seven transmembrane-spanning receptors that represent targets for over 50% of drugs available on the market [1]. These receptors signal through the activation of a heterotrimeric G protein complex, consisting of G α, β, and γ subunits. Upon activation of the receptor, bound guanosine-diphosphate (GDP) is exchanged for guanosine-triphosphate (GTP) in the Gα subunit. This causes a dissociation of the Gα subunit from both the receptor and Gβω subunit complex, and both the Gα subunit and the Gβγ complex proceed to activate their respective signaling pathways. The signal is terminated by the hydrolysis of GTP to GDP in the Gα subunit [2]. The intrinsic, relatively slow rate of hydrolysis of the Gα subunit is temporally modulated by another superfamily of proteins, regulators of G-protein signaling (RGS) proteins, that increases the GTPase rate of a variety of Gα subunits, thus acting as GTPase activating proteins (GAPs) [3].

Due to their important role in regulating GPCR signaling, RGS proteins represent intriguing targets for drug development. In developing high-throughput screening (HTS) assays for RGS targets, methods have emerged for the targeting of the RGS-Gα protein-protein interaction, such as flow cytometry, Alpha Screen, fluorescence polarization, and time-resolved fluorescence resonance energy transfer [4,5,6,7]. These methods have been successfully used to detect the disruption of the protein-protein interaction and not the GAP functionality of the RGS proteins. The predominant method for determination of RGS protein activity has been the use of ³²P labeled GTP in single turnover or steady-state assays [8,9]. While these ³²P assays provide a measure of RGS activity on GTPase activity, they are technically challenging, even in low throughput benchtop experiments which involve the use of radioactivity and require careful timing for reproducible results [10]. Furthermore, assays utilizing entire receptor/protein complexes contained within phospholipid vesicles have proven to be laborious and have not been amenable to high throughput screening [11].

In order to develop a viable HTS assay for measuring GAP function, two hurdles are to be overcome. First, the catalytic activity of the Gα subunit must be slowed to allow for a larger time window. Second, the rate-limiting step of Gα subunit turnover must be shifted from GDP dissociation to GTP hydrolysis. Analysis of the Gα subunit resulted in the previous reports describing a point mutation at the catalytically critical arginine residue (R178C in Gαᵢ₁) that results in a marked reduction in the intrinsic GTPase activity of the Gα subunit while maintaining sensitivity to the GAP activity of RGS proteins [8,12, 13]. Another point mutation, A326S in Gαᵢ₁, allows for a ˜25 fold increase in k_{off(GDP)} while maintaining normal GTPase activity [14, 15]. These two point mutations have been used in the development of another HTS assay, the Transcreener assay (BellBrook Labs; Fitchburg, Wis.) to detect GDP generation [6]. The Transcreener assay relies on the usage of antibodies for the detection of generated GDP by fluorescence polarization. While this assay is well validated and commercially available, the use of antibodies in HTS assays can become prohibitively expensive.

Given the limitations of these approaches, there remains a need to develop simple, non-radioactive assays to measure RGS protein GAP function as well as modulators thereof.

### SUMMARY OF THE INVENTION

In an aspect of the invention there is provided modulators of G protein signaling selected from the group consisting of formula UI-5, UI-1590, UI-1907 and UI-2034:

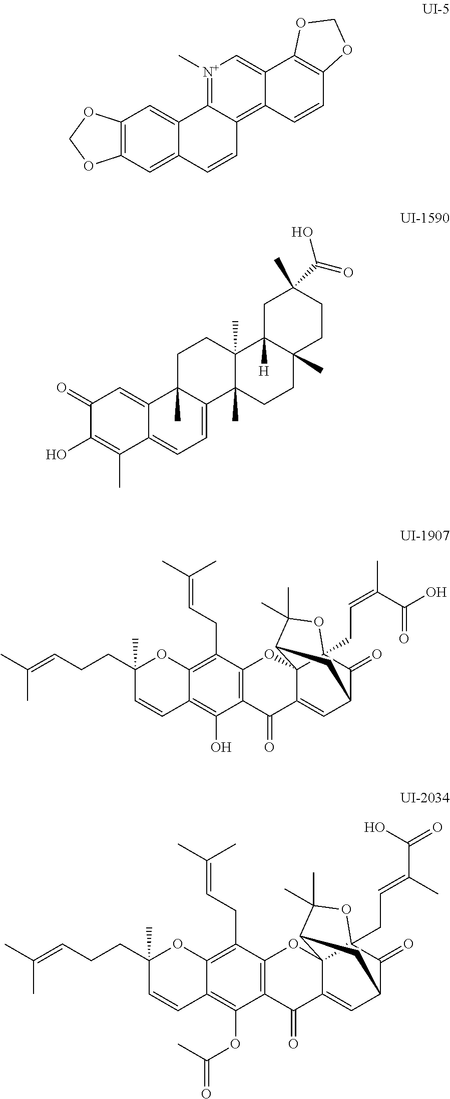

In another aspect of the invention there is provided a method of treating or preventing a disorder mediated by aberrant G protein signaling comprising administering to a patient in need thereof a therapeutically effective amount of a compound selected from the group consisting of formula UI-5, UI-1590, UI-1907 and UI-2034.

In yet another aspect of the invention there is provided a method for identifying modulators of G protein signaling comprising:
contacting an RGS protein with a Gα subunit of a G protein in a solution in the presence of GTP and the presence or absence of test compound,
reacting free phosphate released from the Gα subunit with molybdate to form a phosphomolybdate complex, and
contacting the phosphomolybdate complex with malachite green;
   whereby a reduction in absorbance at about 642 nm and/or about 436 nm in the presence of test compound compared to the absence of the test compound indicates the test compound is an activator of G protein signaling.

In another aspect of the invention there is provided a method for identifying a compound capable of activating a G protein comprising:
contacting an RGS protein with a Gα subunit of a G protein in a solution in the presence of GTP and the presence or absence of test compound,
reacting free phosphate released from the Gα subunit with molybdate to form a phosphomolybdate complex, and
contacting the phosphomolybdate complex with malachite green;
   whereby a reduction in absorbance at about 642 nm and/or about 436 nm in the presence of test compound compared to the absence of the test compound indicates the test compound is an activator of the G protein.

In another aspect of the invention there is provided a method for identifying modulators of an RGS protein comprising:
contacting an RGS protein with a Gα subunit of a G protein in a solution in the presence of GTP and the presence or absence of test compound,
reacting free phosphate released from the Gα subunit with molybdate to form a phosphomolybdate complex, and
contacting the phosphomolybdate complex with malachite green;
   whereby a reduction in absorbance at about 642 nm and/or about 436 nm in the presence of test compound compared to the absence of the test compound indicates the test compound is an inhibitor of the RGS protein signaling.

### BRIEF DESCRIPTION OF THE FIGURES

FIG. 1—Scheme of Malachite Green Assay. RGS protein interacts with Gαᵢ and induces the hydrolysis of GTP to GDP, releasing free phosphate. In the presence of acid, molybdate releases water and complexes with the free phosphate. Lastly, the phosphomolybdate complex associates with the malachite green to produce a strong absorbance peak at 642 nm.

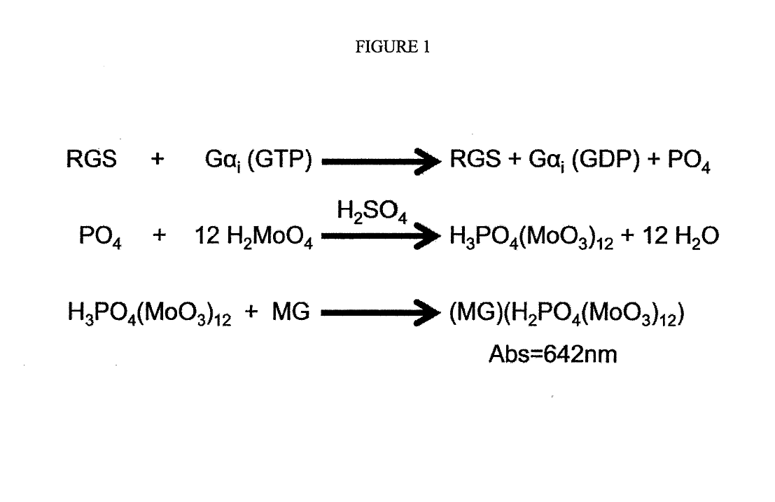

FIG. 2—Optimization of Malachite Green Assay for RGS4. (A) Increasing concentrations of Gαᵢ, 1 μM to 20 μM final, reveals a concentration dependent increase in background signal (opened symbols). The increasing concentration of Gαᵢ also correlates to an increased rate, indicating the measurement of the Gαᵢ intrinsic GTPase rate. Higher concentrations of Gαᵢ similarly result as well as increase of initial rates of RGS4 (200 nM final) stimulated GTPase activity (closed symbols). Comparison of the RGS4 containing to RGS4 null counterpart, the largest signal window was chosen: 5 μM Gαᵢ. (B) Increasing concentrations of RGS4, from 50 to 400 nM final, corresponds to increasing rates of free phosphate generation, closed symbols. In order to maintain the largest dynamic range, ideal concentrations and time points were chosen only in the linear portion of each curve. Secondly, the overall signal was considered when compared to the RGS4 null control, open symbols. This result led to the selection of 200 nM RGS4 as the optimal final concentration and an incubation time of 75 min. (C) Increasing concentrations of the GTP from 50 to 600 μM final resulted in progressively high background. The background subtracted comparison of RGS4 (200 nM final) containing samples (closed symbols) showed little increase in signal across the GTP concentration range while providing a notable increase in Gαᵢ only samples (open circles). The ideal concentration of GTP was chosen to be 150 μM by comparing each signal window.

FIG. 3-Characterization of Malachite Green Assay with RGS8 and RGS17. (A) Increasing concentrations of RGS8 from 5 nM final to 200 nM final, represented as closed symbols, show signal about equal to 2× the concentration of RGS4, similar to as shown in literature [4]. For comparison, 5 μM final Gαᵢ was included, represented by open symbols. (B) Using a Gαᵢ double mutant protein with an accelerated K_{off} for GDP exchange and decrease K_{cat} for GTPase activity we can monitor the effect of RGS 17 on the intrinsic GTPase activity of the Gαᵢ subunit.

FIG. 4—Determination of the Z-factor for 384-well and 1536-well assay. (A) In a 384-well plate, 192 wells were used as a negative control (buffer only), represented by closed circles. An additional 192 wells were used as positive controls and were treated with CCG-50014, a potent RGS4 inhibitor, (10 μM final) represented by open circles [29]. The solid lines represent the mean value for the negative control and the positive control (1.74 and 0.92 respectively). The dashed lines marks the 3 standard deviation cut off for both the positive and negative control (standard deviation of 0.033 and 0.021 respectively). (B) This assay was conducted in 5.5% DMSO to mimic the actual concentration of DMSO in the pilot screen. In a 1536-well plate, 128 wells received buffer, negative control (closed symbols) and the remaining 128 wells received 10 μM final CCG-50014, positive control (open symbols). The solid lines represent the mean value for the negative control and the positive control (0.67 and 0.30 respectively). The dashed lines marks the 3 standard deviation cut off for both the positive and negative control (standard deviation of 0.028 and 0.021 respectively).

FIG. 5—Screen of Spectrum Library. Solid line represents mean negative control. Dashed line represents 3 standard deviations from control and consideration as a hit. (A) In plate one, 16 compounds were identified as initial hits. (B) In plate 2, an additional 43 compounds were identified as initial hits.

FIG. 6—Single Point Hit Confirmation and Control Screens. (A) Single point hit confirmation assay was an analysis of each of the initial hits in a 384-well format (40 μM final for each compound). 7 compounds fell within 3 standard deviations of the negative control and were excluded from further analysis. (B) Phosphate control assay was a comparison of each compound's (40 μM final) ability to inhibit the assay itself, containing 50 μM phosphate instead of protein. Dashed line represents 3 standard deviations from the negative control. 1 compound fell below 3 standard deviations and was excluded from further analysis. (C) At 40 μM final for each compound, the Gαᵢ control assay evaluated each compound for inhibition of Gαᵢ (5 μM final). The dashed line represents 3 standard deviations below the negative control. 5 compounds fell below 3 standard deviations and were excluded from further analysis. Filled bars represent compounds carried over to following experiments. Open bars represent compounds excluded from further analysis.

FIG. 7—ALPHA-Screen orthogonal assay and RGS4(Δ7) counter screen. (A) At 40 μM final for each compound, this assay was used to confirm each compound as an inhibitor of RGS4 (20 nM final) through another assay. The dashed line represents the cutoff, 3 standard deviations from negative control. 15 compounds fell within 3 standard deviations of the negative control and were excluded from further analysis. (B) This single point assay, at 40 μM compound, was used to confirm activity of each compound against the RGS4(Δ7) mutant (200 nM final). The dashed line represents 25% inhibition, the cutoff for compounds carried to dose-response analysis. 18 compounds failed to inhibit the RGS4(Δ7) mutant of RGS4 and were excluded from further analysis. Filled bars represent compounds carried over to following experiments. Open bars represent compounds excluded from further analysis.

FIG. 8-Dose-response analysis of UI-5, UI-1590, UI-1907, UI-2034. (A) Increasing concentrations of compound challenged against RGS4(WT), 200 nM final, in the malachite green assay. (B) The same compounds were compared against the RGS4(Δ7) mutant. All compounds have marked lower potency against the RGS4(Δ7) than the RGS4(WT).

FIG. 9—Structure of identified Compounds. (A) UI-5, also known as sanguinarium sulfate. (B) UI-1590 is the pre-therapeutic anti-cancer compound celastrol [38]. (C) UI-1907 is gambogic acid. (D) UI-2034, acetyl-isogambogic acid, is an analogue of UI-1907.

FIG. 10-Pilot screen results for Spectrum Library. From the 2320 compound library, 59 compounds (2.5%) were considered hits. 52 of those compounds were confirmed in a single point assay. 6 compounds were found to inhibit either the assay or the Gαᵢ directly, leaving 46 compounds (2.0%). An additional 15 compounds were removed for failing the ALPHA Screen orthogonal assay. And finally, 18 compounds were found to not inhibit RGS4(Δ7) at least 25% in the single point counter screen, leaving 13 compounds (0.6%).

### DETAILED DESCRIPTION OF THE INVENTION

RGS proteins are interesting targets due to their role in modulating G-protein signaling. Previous work identifying inhibitors of R4 family RGS proteins have centered on the disruption of the high affinity RGS-Gα interaction observed in the presence of AlF₄⁻, which mimics the transition state of GTP bound to a Gα subunit [4,5]. While valid methods for determination of RGS inhibitors, the transition state mimic generated by AlF₄⁻ generates an RGS-Gα protein:protein interaction with approximately 50-fold higher than basal affinity [31, 32]. The objective of developing this assay was to generate an assay for measuring steady state protein activity that would be economical, fast, easy to use, and adaptable to members of other RGS protein families. The assay developed met each of those criteria.

The initial setup for the assay, for each 1536-well plate, was 1.5 h, which includes incubation steps for the production of free phosphate, allowing the assay to be conducted in highly parallel fashion. Using a colorimetric dye for readout is straightforward and can be accomplished on the simplest of plate readers in absorbance mode. Speed is also essential, and the total read time for each 1536-well plate was only 8 minutes, though this is plate-reader dependent. Perhaps most important is that this assay ameliorates a major concern in high throughput screening—the presence of library compounds that may absorb at a wavelength critical for the assay's readout. In the case of this malachite green assay, the primary wavelength for the absorption read of the assay is at 642 nm, however, a secondary peak is also present at 436 nm, which provides a second readout to help discriminate compounds that may interfere with the primary readout at 642 nm. The absorbance at 436 nm is lower intensity than that at 642 nm, however it is quite usable as a secondary, confirmatory readout—and one that can be run on the same sample as the primary read.

In this study, we developed a malachite-based assay to measure GAP activity of a variety of RGS proteins. RGS4 was selected as the pilot RGS for this assay due to the results of recent RGS4 HTS campaigns and the availability of a small collection of control compounds [4, 6, 7, 20, 21, 22]. While the majority of known RGS4 inhibitors act as irreversible cysteine modifiers (particularly at CYS 148), our group, and others, seek the development of non-covalent RGS inhibitors [23]. The development of reversible inhibitors of RGS4 is of particular interest to the study of Parkinson's disease (PD). Recent research has shown that RGS4 induction is an integral component of the progression of motor symptoms in mouse models of PD [24]. For this reason, in the development of the assay we include a counter screen against the cysteine null mutant of RGS4 (designated Δ7) to eliminate compounds that modify free thiols as their mode of inhibition [21]. This malachite green based assay allowed us to perform steady state analysis of RGS4, RGS8 and RGS17 activity readily in a plate based assay, acquiring data in as little as 40 min, with stability out to 2 h. After development, the absorbance remains stable for at least 30 min after, allowing for multiple reads of the same plate, such as scanning the fainter peak at 405 nm in order to evaluate compounds with strong absorbance at the principle peak of 642 nm [19]. Another benefit of this assay is the negligible cost of performing this assay, at approximately $0.005/well.

Accordingly, in an aspect of the invention there is provided a method for identifying modulators of G protein signaling comprising:
contacting an RGS protein with a Gα subunit of a G protein in a solution in the presence of GTP and the presence or absence of test compound,
reacting free phosphate released from the Gα subunit with molybdate to form a phosphomolybdate complex, and
contacting the phosphomolybdate complex with malachite green;
   whereby a reduction in absorbance at about 642 nm and/or about 436 nm in the presence of test compound compared to the absence of the test compound indicates the test compound is an activator of G protein signaling.

In another aspect of the invention there is provided a method for identifying a compound capable of activating a G protein comprising:
contacting an RGS protein with a Gα subunit of a G protein in a solution in the presence of GTP and the presence or absence of test compound,
reacting free phosphate released from the Gα subunit with molybdate to form a phosphomolybdate complex, and
contacting the phosphomolybdate complex with malachite green;
   whereby a reduction in absorbance at about 642 nm and/or about 436 nm in the presence of test compound compared to the absence of the test compound indicates the test compound is an activator of the G protein.

In another aspect of the invention there is provided a method for identifying modulators of an RGS protein comprising:
contacting an RGS protein with a Gα subunit of a G protein in a solution in the presence of GTP and the presence or absence of test compound,
reacting free phosphate released from the Gα subunit with molybdate to form a phosphomolybdate complex, and
contacting the phosphomolybdate complex with malachite green;
   whereby a reduction in absorbance at about 642 nm and/or about 436 nm of the solution in the presence of test compound compared to the absence of the test compound indicates the test compound is an inhibitor of the RGS protein signaling.

In a particular embodiment, in the foregoing methods the RGS protein, the Gα subunit of a G protein, molybdate and malachite green are added to the same solution. In a particular embodiment, the GTP is added to the solution after the RGS protein, the Gα subunit of a G protein, molybdate and malachite green. In a particular embodiment, the malachite is added to the solution prior to the RGS protein and the RGS protein is added prior to the Gα subunit of a G protein and the Gα subunit of a G protein is added to the solution prior to the GTP.

In a particular embodiment, absorbance at 642 nm and/or 436 nm of a solution described in the foregoing methods containing the test compound is compared with a solution not containing the test compound. In a particular embodiment, a test compound is an activator of G protein signaling when such a solution containing the test compound exhibits reduced absorption at 642 nm compared to such a solution without the test compound (i.e. the method performed in the absence of the test compound). In a particular embodiment, a test compound is an activator of G protein signaling when such a solution containing the test compound exhibits reduced absorption at 436 nm compared to such a solution without the test compound (i.e. the method performed in the absence of the test compound).

RGS protein are a highly diverse protein family, which have unique tissue distributions and functions. RGS proteins can be divided into 8 subfamilies and share a conserved “RGS box” domain (DeVries et al., Proc Natl Acad Sci USA, 1995, 92: 11916). The present invention is not limited to the inhibition of a particular RGS protein. RGS proteins are classified by the type of G protein that they interact with (e.g., including, but not limited to, Gᵢ, Gₒ, G_{q}, Gₜ, G_{z}, and G_{12/13} coupled RGS proteins). Alternatively, RGS proteins are classified based on sub family (Zheng et al., Trends Biochem. Sci., 1999, 24:411). In a particular embodiment, the RGS protein is RGS4. In another embodiment, the RGS protein is RGS8. In another embodiment, the RGS protein is RGS 17. In a particular embodiment, the Gα subunit of a G protein is G_{αi}. In a particular embodiment, the Gα subunit of a G protein is G_{αo}. In a particular embodiment, the Gα subunit of a G protein is G_{αq}. In a particular embodiment, the Gα subunit of a G protein is G_{αt}. In a particular embodiment, the Gα subunit of a G protein is G_{αs}. In a particular embodiment, the Gα subunit of a G protein is G_{αz}. In a particular embodiment, the Gα subunit of a G protein is G_{α12/13}.

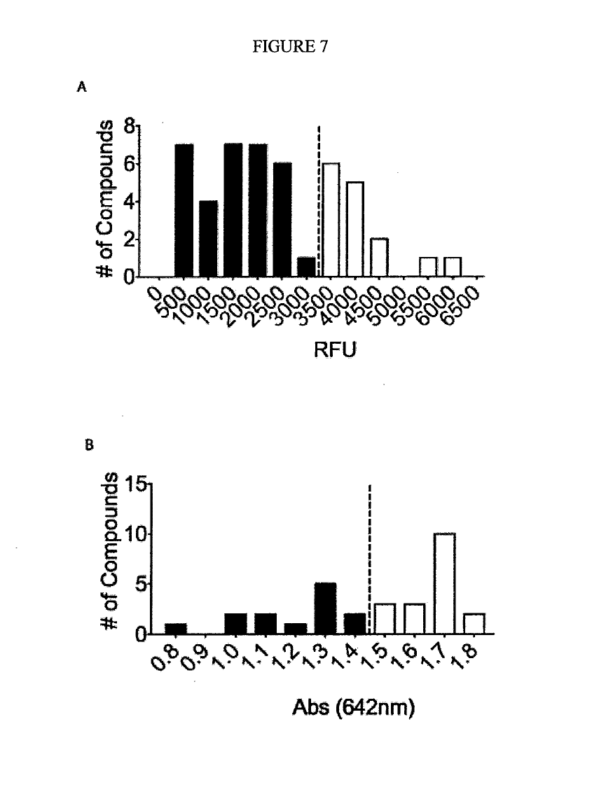

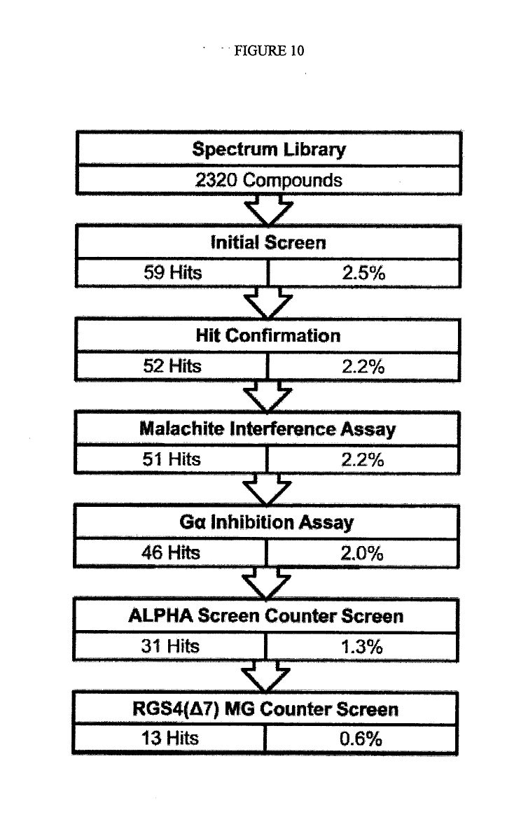

After careful characterization of the constraints of the assay itself, a small-scale, proof-of-concept screen was performed using a small molecule library of 2320 compounds (MicroSource; Gaylordsville, Conn.), summarized in FIG. 10. The initial results for the 2320 compound library yielded an initial hit rate of 2.5% (59 compounds) that inhibited (by at least 3 standard deviations below the negative control) RGS-mediated GAP activity. RGS-mediated GAP activity is indicated by an increase in free Pᵢ, generated by hydrolysis of GTP, available to complex with malachite green and increase absorbance at 642 nm. An initial triage included the exclusion of hit compounds that interfered with the assay by directly inhibiting the chemical reactions of the assay readout or inhibiting Gαᵢ itself reduced this hit rate to approximately 2.0%. 7 compounds failed to inhibit RGS4 greater than 3 standard deviations from the negative control in the initial hit confirmation assay using 40 μM compound. 1 compound was found to interfere with the malachite green assay directly, as shown when challenged in an assay containing only 50 μM PO₄, (greater than 3 standard deviations from the negative control). Finally, an additional 5 compounds were found to inhibit the intrinsic GTPase activity (greater than 3 standard deviations from the negative control) of the Gαᵢ subunit alone. A second, confirmatory screen of the initial hit compounds was performed using an orthogonal assay, ALPHA Screen (Perkin Elmer; Waltham, Mass.), further reduced this to a hit rate of 1.3% [5]. A Single point ALPHA Screen, using the same concentration as the initial screen, eliminated an additional 15 compounds that failed to inhibit at least 3 standard deviations from the positive control. Of the 31 compounds only 13, 0.6% of all compounds screened, were shown to inhibit the RGS4(Δ7) construct (FIG. 7B) greater than 25% from the negative control. The RGS4(Δ7) mutant was used as a filter in order to avoid thiol-modifiers similar to compounds already identified previously [22, 23]. These compounds identified in the screen described here were shown weaker inhibitors of the RGS4(Δ7) mutant versus the wild type construct with the exception of two compounds, UI-587 and UI-992.

Each of the compounds demonstrates inhibition of RGS4. Some of the more potent compounds identified, such as UI-587 and UI-662, contain covalent cysteine and free amine chemical functionalities similar to those that have been discovered in other screens against RGS4 [22,23]. Interestingly, two very similar compounds, UI-1907 and UI-2034, were determined to be inhibitors of RGS4 and the RGS4(Δ7). Also identified in this screen is a series of compounds with a quinone functionality, UI-1775, UI-1925, UI-2144, UI-2202, UI-2231, and UI-2249. One of these compounds was the most potent inhibitor of the RGS4(Δ7) mutant, UI-2144. With IC₅₀ values from 20-30 μM, UI-1775, UI-2144, UI-2202, and UI-2249 represent some of the most potent compounds reported for the RGS4(Δ7) mutant [4]. Certain compounds, UI-587 and UI-992, inhibited both RGS4 and the RGS4(Δ7) mutant equally. We expected UI-587 to inhibit both equally due to its potential mechanism of action including the modification of free amines. Several of the compounds identified in this screen represent interesting structures, such as UI-5 and UI-1590, and warrant additional investigation, as their mode of action in inhibiting RGS4 is not readily apparent.

Compounds targeting RGS proteins include, but are not limited to: 1) potentiators of endogenous agonist function, 2) potentiators/desensitization blockers of exogenous GPCR agonists, 3) specificity enhancers of exogenous agonists, 4) antagonists of effector signaling by an RGS protein, and 5) RGS agonists (see e.g., Zhong and Neubig, Journal of Pharmacol. and Exp. Ther., 2001, 297:837). It is contemplated that inhibitors of the present invention inhibit GTPase acceleration activity of RGS proteins. Accordingly, in an aspect of the invention there is provided compounds that inhibitor RGS protein mediated acceleration of G protein GTPase activity and/or RGS protein binding to G protein or subunits thereof wherein said in said compounds are selected from the group consisting of UI-5, UI-1590, UI-1907 and UI-2034:

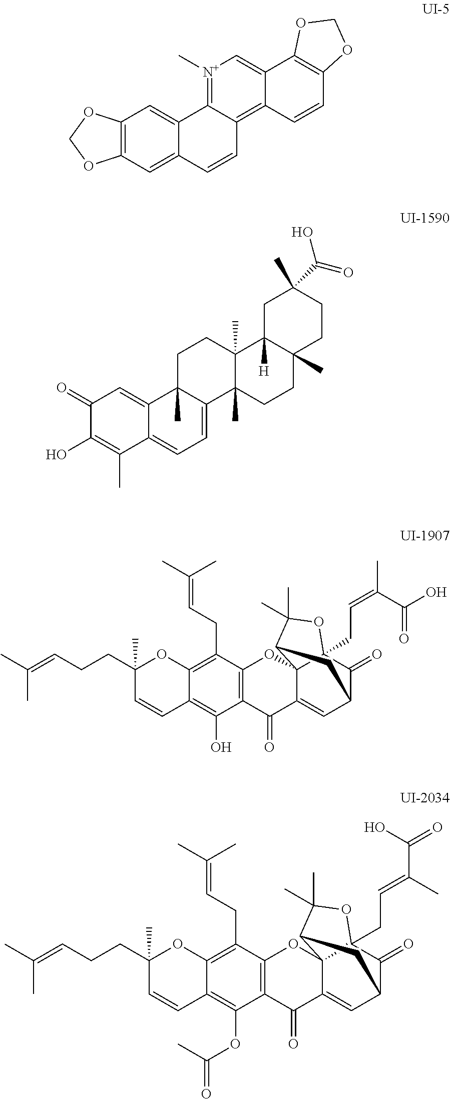

In another aspect of the invention there is provided modulators of G protein signaling selected from the group consisting of formula UI-5, UI-1590, UI-1907 and UI-2034. In another aspect of the invention there is provided a method of treating or preventing a disorder mediated by aberrant G protein signaling comprising administering to a patient in need thereof a therapeutically effective amount of a compound selected from the group consisting of formula UI-5, UI-1590, UI-1907 and UI-2034. In a particular embodiment, the disease is a Parkinson's disease comprising administering to said patient a therapeutically effective amount of a compound selected from the group consisting of UI-5, UI-1590, UI-1907 and UI-2034.

The compounds of the invention, may be prepared using established organic synthetic techniques from commercially available starting materials. Alternatively, the compounds may be commercially available.

Another aspect of the invention provides pharmaceutical compositions or medicaments containing the compounds of the invention and a therapeutically inert carrier, diluent or excipient, as well as methods of using the compounds of the invention to prepare such compositions and medicaments. In one example, the compounds may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, the compounds are formulated in an acetate buffer, at pH 5. In another embodiment, the compounds are sterile. The compounds may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit RGS protein activity. For example, such amount may be that required to prevent RGS protein from deactivating G protein.

In one example, the pharmaceutically effective amount of the compounds of the invention administered parenterally per dose will be in the range of about 0.001 to 1,000 (e.g., 0.01-100) mg/kg, alternatively about 0.05 to 50 (e.g., 0.1 to 20) mg/kg of patient body weight per day, with the typical initial range of the compounds used being 0.3 to 15 mg/kg/day. In another embodiment, oral unit dosage forms, such as tablets and capsules, preferably contain from about 0.1 to about 1,000 (e.g., 25-100) mg of the compounds of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 1 to 1,000 (e.g., 25 mg, 50 mg, 100 mg, 250 mg, or 500 mg) of the compounds of the invention compounded with about 1 to 1,000 (e.g., 90-300) mg anhydrous lactose, about 1 to 100 (e.g., 5-40) mg sodium croscarmellose, about 0.1 to 100 (e.g., 5-30 mg) polyvinylpyrrolidone (PVP) K30, and about 0.1 to 100 (e.g., 1-10 mg) magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compounds, for example 1 to 1000 (e.g., 5-400 mg), of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of the invention, or a stereoisomer or pharmaceutically acceptable salt thereof. In a further embodiment includes a pharmaceutical composition comprising a compound of the invention, or a stereoisomer or pharmaceutically acceptable salt thereof, together with a pharmaceutically acceptable carrier or excipient.

Another embodiment includes a pharmaceutical composition comprising a compound of the invention for use in the treatment of Parkinson's disease. Another embodiment includes a pharmaceutical composition comprising a compound of the invention for use in the treatment of cancer.

### EXAMPLES

The invention will be more fully understood by reference to the following examples. They should not, however, be construed as limiting the scope of the invention.

### Example 1

### Expression and Purification of Recombinant Protein

Tobacco etch virus (TEV) protease was expressed and purified as a His-tagged protein in E. coli, BL21-pRIL (Stratagene; La Jolla, Calif.), in the pRK793 vector as previously described by the Waugh lab [25].

Rat RGS4, sharing 97% sequence identity with human RGS4, and the cysteine to alanine mutant were expressed as fusion proteins of maltose binding protein (MBP), a 10× His tag, and a TEV protease recognition site fused to the N-terminus of an RGS4 construct containing amino acids 51-205, in the vector pMALC2H10T in BL21-DE3 bacteria (Stratagene; Santa Clara, Calif.) [26]. The single cysteine-null Δ51-RGS4 construct was generated by site-directed mutagenesis as described previously [23] Expression and purification were performed as described previously [23]. Purified protein was incubated with TEV protease at a molar ratio of 10:1 (fusion protein:TEV protease) overnight at 4° C. The cleaved Δ51-RGS4 was then isolated by purification over an ANX column (GE Healthcare; Fairfield, Conn.) in 50 mM HEPES at pH6.8 and 50 mM NaCl. The flow through, containing the ˜99% Δ51-RGS4 as determined by SDS-PAGE gel, was then collected and concentrated using a YM-10 centrifugal concentrator (Millipore; Billerica, Mass.). The concentration of Δ51-RGS4 was calculated based on the absorbance at 280 nm utilizing a Take-3 plate (Biotek; Winnoski, Vt.) in a Synergy 2 plate reader (Biotek; Winnoski, Vt.).

Human RGS8 expression and purification was performed similar to other RGS8 purifications previously reported [27]. An RGS8 truncated construct analogous to the RGS4(Δ51) construct described above, amino acids 60-198 with a C-terminal 6× His tag in the pET28 vector was expressed in BL21-RIPL (Stratagene; Santa Clara, Calif.) cells cultured in Terrific Broth (TB) media. Cultures were induced with 200 μM IPTG at OD_{600 nm of} 2.0 and cultured for 16 h at 18° C. Pellet was lysed, centrifuged, and filtered as described above except in RGS8 Buffer (50 mM HEPES at pH 7.5, 500 mM NaCl, 0.5 mM β-mercaptoethanol). Samples were loaded onto a Ni-NTA column (Qiagen; Hilden, Germany), 3 mL for every 1 L media, and washed with RGS8 Buffer supplemented with 25 mM imidazole. The protein was eluted using 200 mM imidazole and fractions were analyzed by SDS-PAGE gel. Fractions containing RGS8>95% purity were pooled and protein concentration was determined by 280 nm absorbance as accomplished above. Human RGS17 was expressed and purified as a His-tagged protein in E. Coli BL21-DE3 (Stratagene; La Jolla, Calif.), in the pET28 vector as previously described [5].

Human Gαᵢ (R178M, A326S) rate-altered variant described in literature, was expressed in TB media as a 6× His labeled protein in the pQE80 vector [6]. Expression was induced, at OD_{600 nm} of 1.0, with 100 μM IPTG at 30° C. for 16 h. Pellets were lysed, centrifuged, and filtered as described above, but in Gαᵢ Buffer (50 mM HEPES at pH 7.5, 500 mM NaCl, 1 mM β-mercaptoethanol, and 20 μM GDP). The sample was first loaded onto a Ni-NTA column (Qiagen; Hilden, Germany), containing 3 mL of resin for every 1 L of media. The column was first washed with Gαᵢ Buffer supplemented with 25 mM imidazole. Gαᵢ was then eluted from the column with Gαᵢ buffer supplemented with 300 mM imidazole. After analysis by SDS-PAGE gel, fractions that contained Gαᵢ were pooled and dialyzed overnight against Gαᵢ Dialysis Buffer (50 mM HEPES at pH7.5, 25 mM NaCl, 1 mM β-mercaptoethanol, and 20 μM GDP). The sample was then loaded onto a Q-sepharose column (GE Healthcare; Fairfield, Conn.) and elutqd along a salt gradient from 50 mM NaCl to 1M NaCl in Gαᵢ Buffer. The resulting peaks were then analyzed by SDS-PAGE for fractions containing >99% Gαᵢ. The purified Gαᵢ was then assayed for activity utilizing the [³⁵S]GTPγS binding assay [28].

Rat Gαₒ was expressed in LB media as a fusion protein of glutathione-S-transferase (GST), 6×His, and Gαₒ, in pQLinkGD vector. Expression was induced, at OD_{600 nm} of 0.5, with 100 μM IPTG at 30° C. for 16 h. Pellets were lysed, centrifuged, and filtered as described above, but in Gαₒ Buffer (50 mM HEPES at pH8, 100 mM NaCl, 10 μM GDP, 1 mM tris(2-carboxethyl)phosphine). The protein was first purified over a nickel charged resin column, 1 mL resin for every 1 L culture. Prior to elution, the column was washed with 20 mM imidazole to clear weak binding contaminants from the sample. The fusion protein was eluted with 250 mM imidazole. Fractions were collected and analyzed by SDS-PAGE gel. Fractions containing the protein of interest were pooled and loaded onto glutathione sepharose column (GE Healthcare; Fairfield, Conn.), 1.5 mL resin for every 1 L culture. The protein was then eluted with 1 mM free glutathione and analyzed by SDS-PAGE gel. Fractions containing >99% pure protein were pooled for activity determination. The purified Gαₒ was then assayed for activity utilizing the [³⁵S]GTPγS binding assay [28].

### Example 2

### Malachite Green Assay

Stock solutions of each of the 3 components of the developing solution were prepared, which were stable for long-term storage [19]. Malachite solution was prepared by first diluting concentrated sulfuric acid 1:5 in distilled water. Once the solution cooled to 25° C., malachite solution was prepared by dissolving 0.44 g of malachite green oxalate (Alfa Aesar; Ward Hill, Mass.) in 360 mL diluted acid and stored at 25° C. Molybdate solution, containing 7.5% ammonium molybdate tetrahydrate (Alfa Aesar; Ward Hill, Mass.), was prepared in distilled water and stored at 4° C. Tween-20 solution, used to maintain solubility of the phosphate-molybdate-malachite complex, was prepared as 11% (v/v) Tween-20 in distilled water. On the day of use, 2.5 mL molybdate solution and 0.2 mL Tween-20 solution were added to 10 mL of malachite solution and mixed quickly to avoid precipitation of malachite. The final ratio of the Developing Solution (DS) was 50:12.5:1 (malachite:molybdate:Tween-20). The peak absorbance was determined by a 2 nm step wavelength scan, using 50 μM NaH₂PO₄ at pH 8.0 as the negative control.

The malachite green assay involved 5 components, with a 1 min spin at 100×g between each addition. For time-course experiments, the first component was 10 μL Malachite Green Assay Buffer (MGB; 50 mM HEPES at pH 7.5, 100 mM NaCl, 5 mM EDTA, 10 mM MgCl₂, 0.01% lubrol) into a clear 384-well plate (ThermoFisher Scientific; Waltham, Mass.) using a MultiDrop dispenser (PerkinElmer; Waltham, Mass.). The second component dispensed was 10 μL of a 4× stock of RGS4, typically 200 nM to 1.6 μM with the target final concentration of 50 nM to 400 nM, diluted in MGB. After a 30 min incubation, 10 μL of the third component, 4× stock of Gαᵢ diluted in MGB, was dispensed (typically between 4 μM and 80 μM with a desired final concentration 1 μM to 20 μM). After a minimum of 5 min incubation, 10 μL of the fourth component, 4×GTP diluted in MGB, was added at 10 minute intervals from 1-110 minutes. 4×GTP concentrations varied between 0.2 mM and 2.4 mM, with a target final concentration of 50 μM to 600 μM. To terminate the reaction, 10 μL of DS was added to each well using a Microlab Star liquid handling robot (Hamilton Robotics; Reno, Nev.), to achieve a final ratio 4:1 (sample:developing solution). Following the spin, the plate was incubated for 25 min before being read at 642 nm for absorbance using an EnVision plate reader (PerkinElmer; Waltham, Mass.). RGS8 was evaluated similarly to as described for RGS4, with 4× stock concentrations from 20 nM and 800 nM.

Time-course experiments for the RGS 17 were conducted using the 5 component mixture, with a 1 min spin at 500×g between each addition. The first component was 10 μL MGB into a clear 384-well plate as previously described. The second component dispensed was 10 μL of a 4× stock of RGS 17 ranging between 1 μM to 4 μM with the target final concentration of 500 nM to 1 μM, diluted in MGB. After a 30 min incubation, 10 μL of the third component, a 4× stock of Gαᵢ₁ diluted in MGB, was dispensed at a concentration of 4 μM into each well with a final target concentration of 1 μM. This was incubated for a minimum of 5 min. Then 10 μL of the fourth component, 4×GTP at 1.2 mM diluted in MGB, was added at 10 min intervals from 1-110 minutes with a final concentration of 300 μM. Reaction was terminated as previously described using 10 μL of DS and absorbance was read at 642 nm.

Malachite green compound activity and Z-factor analysis conducted in 384-well plates utilized optimized parameters as discerned from the time-course experiments. 10 μL of 4× compound or MGB was dispensed into appropriate wells. For single point assay, 160 μM compound was used, and for dose-response assays a series of ½ log dilutions from 100 μM final to 316 μM final was used. 10 μL of the optimized 4×RGS4 concentration, 0.8 μM in MGB, was dispensed into all wells. After a spin down at 100×g for 1 min, the assay plate was incubated at 25° C. for 30 min. 10 μL of the optimized Gαᵢ concentration, 20 μM in MGB, was dispensed to each well and incubated at 25° C. for 5 min. 10 μL of the optimized 4×GTP, 600 μM in MGB, was then added to the samples. After spinning the samples down at 100×g for 1 min, the samples were incubated at 25° C. for 75 min. The samples were then stamped with 10 μL of DS and incubated for 25 min before reading absorbance at 642 nm.

1536-well Z-factor analysis and compound library screen were accomplished largely as described for 384-well plates. Initial screen and Z-factor determination was performed in a final concentration of 5.5% dimethylsulfoxide. For 1536-well assays NUNC clear plates were used (ThermoFisher Scientific; Waltham, Mass.). Each component was dispensed as 1.8 μL samples into each well using a FlexDrop (PerkinElmer; Waltham, Mass.). To develop the plates, 1.8 μL of DS were stamped in quadrants using the Microlab Star liquid handling robot. After a 25 min incubation, the plates were analyzed using an EnVision plate reader (PerkinElmer; Waltham, Mass.) at 642 nm absorbance.

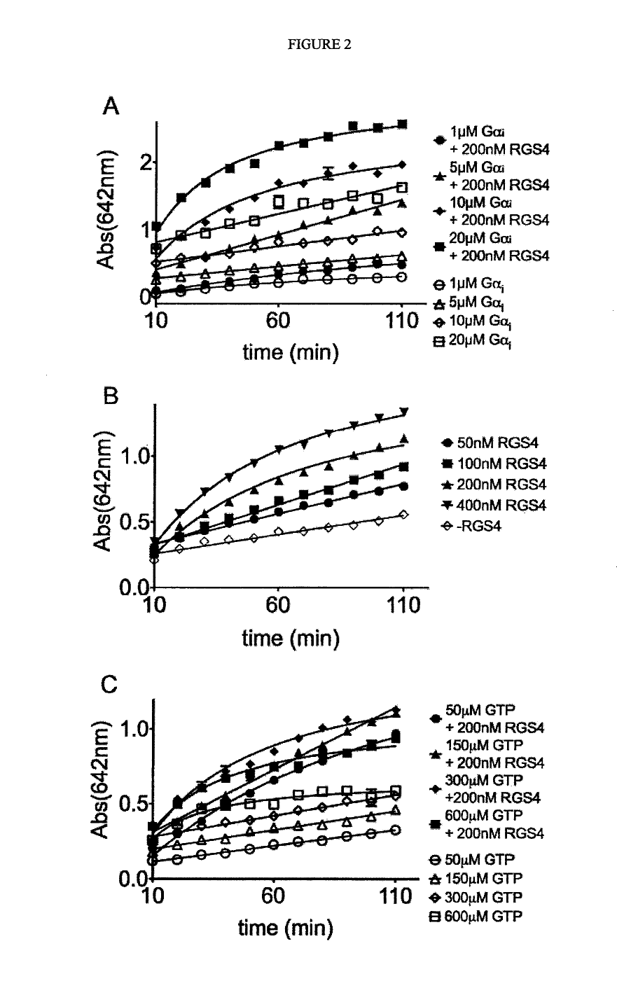

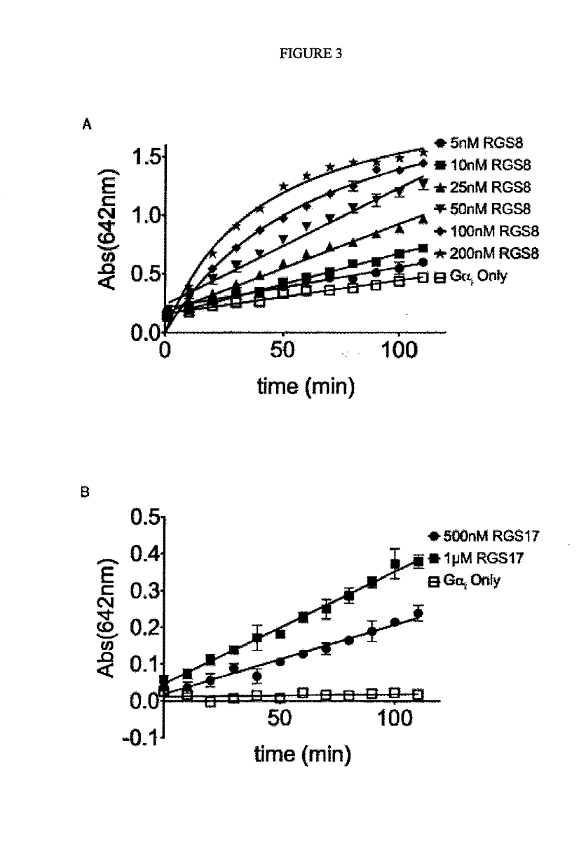

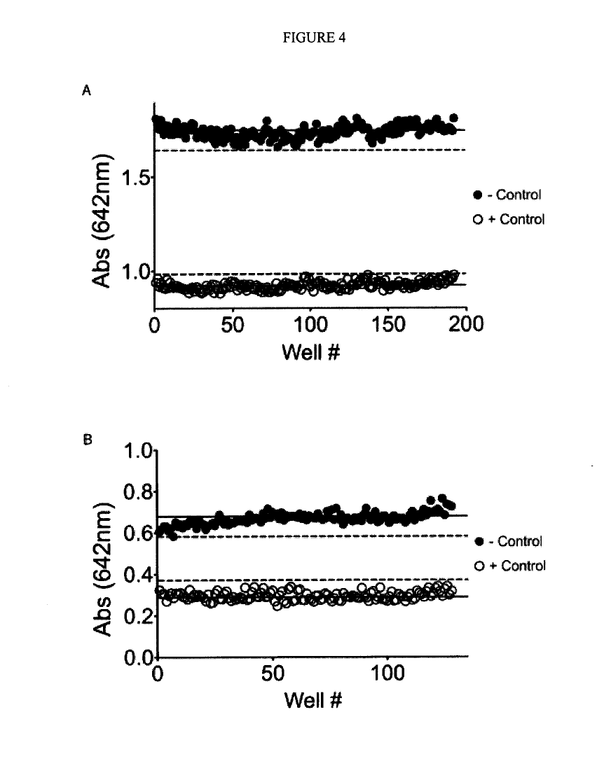

The initial focus of these experiments was to determine optimal conditions for the malachite green assay. A wavelength scan of 40 μL of 50 μM NaPO4 developed for 50 min with 10 μL DS yielded an intense signal peak at 642 nm, with a secondary peak at 436 nm (data not shown). This coincides closely with the reported literature values of 642 nm and 436 nm [19]. Initial concentrations for each of the components were determined as a ratio similar to previously reported values [6]. 5 μM Gαi was chosen due to the similar signal window generated at later time points, when compared to the earlier time points of higher concentrations (both 10 and 20 μM Gαi), and significantly greater signal window than the lower concentration of Gαi, as shown in FIG. 2A. At higher concentrations of Gαi, a basal level of GTPase activity was detectable from the Gαi alone (FIG. 2A). Having selected a Gαi concentration of 5 μM, variable concentrations of RGS4 were compared, as shown in FIG. 2B, with 200 nM RGS4 selected as the optimal concentration due to the higher signal while maintaining linearity up to 80 min. Varying GTP concentration, as shown in FIG. 2C, revealed background noise introduced by the GTP stock, due to residual Pi from GTP hydrolyzed during storage. Conversely, too little GTP showed substrate depletion early in the assay. For use in determining the quality of the assay, the final concentrations of 200 nM RGS4, 5 μM Gαi, and 150 μM GTP were chosen. For comparison, various RGS8 concentrations were challenged against the optimized Gαi and GTP concentrations, (FIG. 3A), and, as previously reported in literature, RGS8 was able to develop a similarly sized signal window with about ½ as much protein [29]. For comparison outside the R4 family, a RZ/A family member, RGS 17, was similarly challenged and, as previously reported in literature, more RGS 17 was required to generate a similar signal window (FIG. 3B) [16]. To confirm the value of this now optimized assay, a comparison of RGS4 with and without inhibitor was used to determine a Z-factor of 0.8, as shown in FIG. 4A.

### Example 2

### ALPHA-Screen Counter-Screen of RGS4

Chemical labeling of RGS4 was performed using biotinamidohexanoic acid N-hydroxy succinimide ester (Sigma Aldrich; St Louis, Mo.). The reaction was carried out at a molar ratio of 3:1 (label/protein) for 3 h at 4° C. in 50 mM HEPES at pH8 and 100 mM NaCl, similar to as previously described [21]. The reaction was then quenched with 10 μL of 1M glycine for 10 min at 4° C. The free label was then separated from the desired protein using a YM-10 centrifugal concentrator. Final concentration of RGS4 was determined by 280 nm absorbance of the sample.

To prepare RGS4 for analysis using the ALPHA-Screen assay, RGS4 constructs were first labeled in a 1440 μL sample, diluted in Assay Buffer (AB 20 mM HEPES at pH8, 100 mM NaCl, 0.1% Lubrol, 1% bovine serum albumin), containing 60 nM RGS4, 14.4 μL streptavidin ALPHA-Screen beads (Perkin-Elmer; Waltham, Mass.). The sample was then incubated for 30 min, on ice, prior to dilution with AB to 2880 μL. In duplicate, 20 μL of each compound at 120 μM was plated across a white 384-well plate (ThermoFisher Scientific; Waltham, Mass.). 20 μL RGS4 was then plated into each well and the samples were incubated at 19° C. for 30 min prior to the addition of GST-Gαₒ. The final concentrations for RGS4 and compound will be 20 nM and 40 μM respectively.

GST-Gαₒ was prepared for the assay by creating a 1440 μL labeling reaction, diluted in AB, containing 3 nM GST-Gαₒ, 10 μM GDP, and 14.4 μL anti-GST ALPHA-Screen Beads (Perkin-Elmer; Waltham, Mass.). The sample was incubated for 30 min on ice. A 40 μL sample was then removed and diluted with 40 μL AB; this is positive control. The remaining 1400 μL is then diluted with 1400 μL AB supplemented with AMF (5 μM AlCl₃, 5 mM MgCl₂, 5 mM NaF) to a final volume of 2800 μL. 20 μL of each sample was then dispensed into the each well. The final concentration of the GST-Gαₒ was be 0.5 nM. Following the addition of both GST-Gαₒ and biotinylated Δ51-RGS4, the plates were incubated at 19° C. for 1 hr prior to reading using the Synergy 2 plate reader. The results were then analyzed and graphed using Prism software (Graphpad Software; La Jolla, Calif.).

### Example 3

### HTS Screen

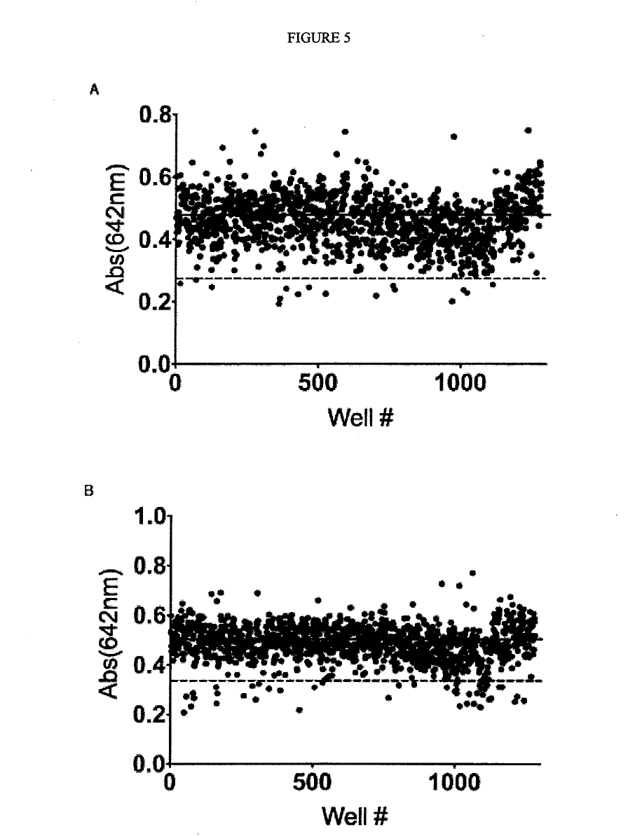

Following initial characterization of the malachite green assay, the assay was optimized for use in a 1536-well HTS format. Maintaining identical concentrations to the development of the assay in 384-well format, the miniaturized assay yielded a Z-factor of 0.6, FIG. 4B. A screen of the Spectrum library was performed in 2 1536-well plates and a final concentration of 40 μM for each compound. Compounds were determined to be hits if they were greater than 3 standard deviations from the mean negative control values. From this initial screen of 2320 compounds, 59 compounds (2.5%) were determined to be hits, FIG. 5A and FIG. 5B. While this would normally be considered an exceedingly high initial hit rate, the Spectrum Library consists of large set of known biologically active compounds [30].

### Example 4

### Hit Confirmation and Counter-Screen

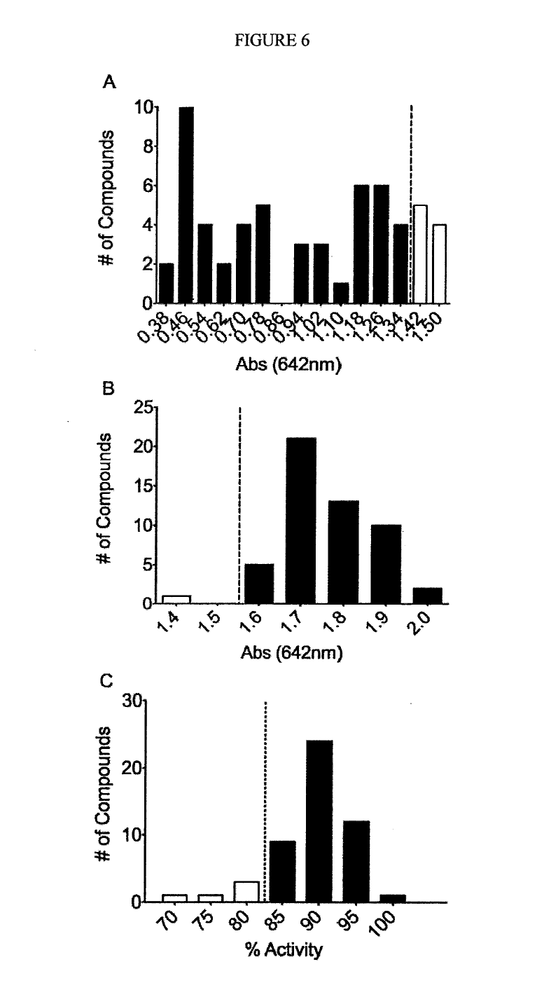

Initial hits were confirmed by single point malachite green assay at 40 μM compound. Of the initial 59 compounds, 7 compounds fell within 3 standard deviations of the negative control, (FIG. 6A), leaving 52 compounds (2.2%). The assay was followed up with an interference assay designed to test for inhibition of the detection method using 50 μM NaPO4 to mimic the maximum detectable released Pi by the assay. This control would detect compounds that either interrupt the detected complex or reduce the molybdate resulting in peak shift outside of the desired wavelength. One compound was found to disrupt the assay (FIG. 6B). Compounds that increased the predicted absorbance were carried through, as they would indicate false negatives in the assay. A counter-screen focusing on the intrinsic GTPase activity of the Gαi mutant followed (FIG. 6C). Utilizing the known GTPase activity of the Gαi mutant, this assay identified compounds that inhibited the Gαi subunit rather than the RGS protein. This assay, conducted at 40 μM compound, identified 5 compounds that interfered with the assay due to the compound falling 3 standard deviations below the negative control, bringing the total to 45 compounds (1.6%) of the screened library. ALPHA Screen was utilized as an orthogonal assay to confirm each of the remaining compounds as hits (FIG. 7A). ALPHA Screen has been successfully used to assay RGS-G-protein interactions in literature [5]. The ALPHA-Screen assay functions by measuring the amount of stable complex formed between the RGS protein and the Gα subunit using the transition state mimic AlF₄⁻. This orthogonal assay eliminated 15 compounds, leaving 30 compounds or 1.3% of the total compounds screened. Finally, compounds were challenged against the RGS4(Δ7) mutant in the malachite green phosphate detection assay, with the desire of eliminating thiol-modifiers similar to those previously discovered in HTS campaigns against RGS4 [23]. Of the 30 compounds remaining, only 13 compounds also inhibited the RGS4(Δ7) mutant (FIG. 7B).

### Example 5

### Characterization of Confirmed Compounds

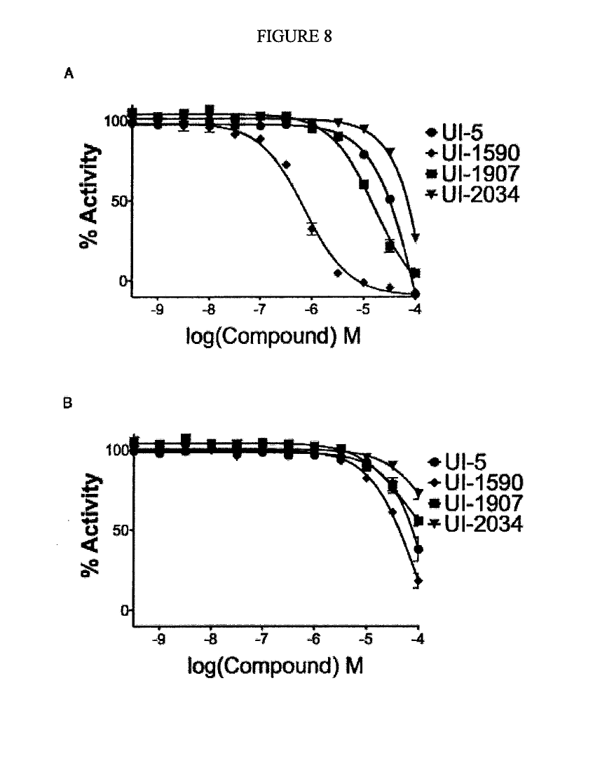

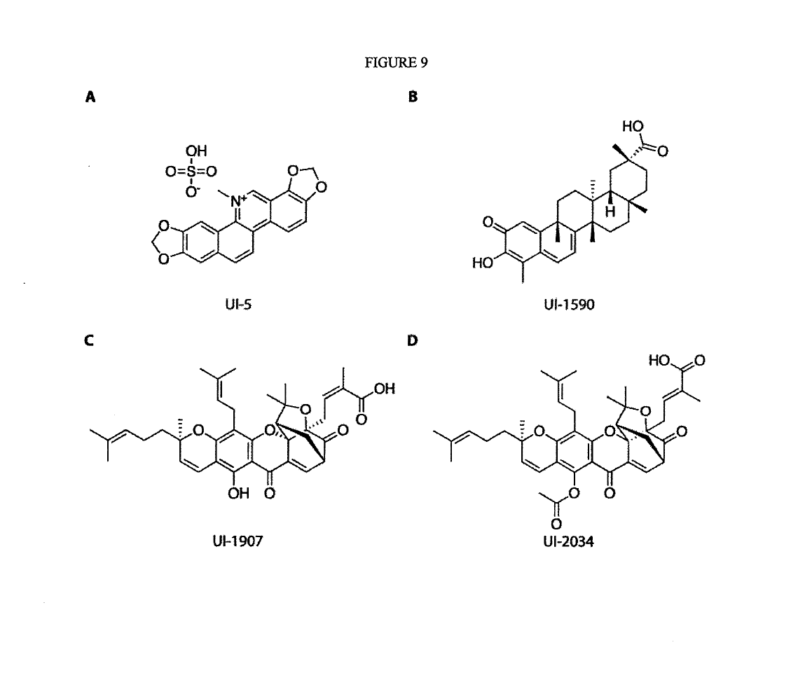

The activity of each of the 13 remaining compounds was assayed by generating concentration-response curves against RGS4 as well as the RGS4(Δ7) mutant. FIG. 8A and FIG. 8B shows the 4 compounds selected for future analysis. UI-5 (FIG. 9A) had an IC₅₀ of 126 μM and 454 μM against the RGS4(WT) and RGS4(47) respectively. The most potent compound, UI-1590 (FIG. 9B), had an IC₅₀ of 724 nM against RGS4(WT) and an IC₅₀ of 88 μM against RGS4(Δ7). Finally, two structurally similar compounds, UI-1907 (FIG. 9C) and UI-2034 (FIG. 9D), had IC₅₀ values of 16 μM and ˜269 mM against RGS4(WT), respectively. Against the RGS4(Δ7) mutant, the compounds had IC₅₀ values of 51 μM and 181 μM, respectively. Each of the hit compounds were far less potent against the RGS4(Δ7) mutant than RGS4(WT), similar to what has been reported in literature [4].

### LIST OF REFERENCES

1. Nürnberg B, Gudermann T, Schultz G (1995) Receptors and G proteins as primary components of transmembrane signal transduction. Journal of Molecular Medicine 73: 123-132.
2. Gilman A G (1987) G proteins: transducers of receptor-generated signals. Annual review of biochemistry 56: 615-649.
3. Berman D M, Gilman A G (1998) Mammalian RGS proteins: barbarians at the gate. The Journal of biological chemistry 273: 1269-1272.
4. Roman D L, Ota S, Neubig R R (2009) Polyplexed flow cytometry protein interaction assay: a novel high-throughput screening paradigm for RGS protein inhibitors. Journal of biomolecular screening 14: 610-619.
5. Mackie D I, Roman D L (2011) Development of a novel high-throughput screen and identification of small-molecule inhibitors of the Galpha-RGS 17 protein-protein interaction using AlphaScreen. Journal of biomolecular screening 16: 869-877.
6. Zielinski T, Kimple A J, Hutsell S Q, Koeff M D, Siderovski D P, et al. (2009) Two Galpha(i1) rate-modifying mutations act in concert to allow receptor-independent, steady-state measurements of RGS protein activity. Journal of biomolecular screening 14: 1195-1206.
7. Blazer L L, Roman D L, Chung A, Larsen M J, Greedy B M, et al. (2010) Reversible, allosteric small-molecule inhibitors of regulator of G protein signaling proteins. Molecular pharmacology 78: 524-533.
8. Berman D M, Wilkie T M, Gilman A G (1996) GAIP and RGS4 are GTPase-activating proteins for the Gi subfamily of G protein alpha subunits. Cell 86: 445-452.
9. Mukhopadhyay S, Ross E M (1999) Rapid GTP binding and hydrolysis by G(q) promoted by receptor and GTPase-activating proteins. Proceedings of the National Academy of Sciences of the United States of America 96: 9539-9544.
10. Chidiac P, Markin V S, Ross E M (1999) Kinetic control of guanine nucleotide binding to soluble Galpha(q). Biochemical pharmacology 58: 39-48.
11. Ingi T, Krumins A M, Chidiac P, Brothers G M, Chung S, et al. (1998) Dynamic regulation of RGS2 suggests a novel mechanism in G-protein signaling and neuronal plasticity. The Journal of neuroscience: the official journal of the Society for Neuroscience 18: 7178-7188.
12. Coleman D E, Berghuis A M, Lee E, Linder M E, Gilman A G, et al. (1994) Structures of active conformations of Gi alpha 1 and the mechanism of GTP hydrolysis. Science 265: 1405-1412.
13. Freissmuth M, Gilman A G (1989) Mutations of GS alpha designed to alter the reactivity of the protein with bacterial toxins. Substitutions at ARG187 result in loss of GTPase activity. The Journal of biological chemistry 264: 21907-21914.
14. Posner B A, Mixon M B, Wall M A, Sprang S R, Gilman A G (1998) The A326S mutant of Gialphal as an approximation of the receptor-bound state. The Journal of biological chemistry 273: 21752-21758.
15. Iiri T, Herzmark P, Nakamoto J M, van Dop C, Bourne H R (1994) Rapid GD P release from Gs alpha in patients with gain and loss of endocrine function. Nature 371: 164-168.
16. Kodama T, Fukui K, Kometani K (1986) The initial phosphate burst in AT P hydrolysis by myosin and subfragment-1 as studied by a modified malachite green method for determination of inorganic phosphate. Journal of biochemistry 99: 1465-1472.
17. Bell R D, Doisy E A (1920) Rapid colorimetric methods for the determination of phosphorus in urine and blood. Journal of Biological Chemistry 44: 55-67.
18. Itaya K, Ui M (1966) A new micromethod for the colorimetric determination of inorganic phosphate. Clinica chimica acta; international journal of clinical chemistry 14: 361-366.
19. Baykov A A, Evtushenko O A, Avaeva S M (1988) A malachite green procedure for orthophosphate determination and its use in alkaline phosphatase-based enzyme immunoassay. Anal Biochem 171: 266-270.
20. Young K H, Wang Y, Bender C, Ajit S, Ramirez F, et al. (2004) Yeast-based screening for inhibitors of RGS proteins. Methods in enzymology 389: 277-301.
21. Roman D L, Talbot J N, Roof R A, Sunahara R K, Traynor J R, et al. (2007) Identification of small-molecule inhibitors of RGS4 using a high-throughput flow cytometry protein interaction assay. Molecular pharmacology 71: 169-175.
22. Roof R A, Roman D L, Clements S T, Sobczyk-Kojiro K, Blazer L L, et al. (2009) A covalent peptide inhibitor of RGS4 identified in a focused one-bead, one compound library screen. BMC pharmacology 9: 9.
23. Roman D L, Blazer L L, Monroy C A, Neubig R R (2010) Allosteric inhibition of the regulator of G protein signaling-Galpha protein-protein interaction by CC G-4986. Mol Pharmacol 78: 360-365.
24. Lerner T N, Kreitzer A C (2012) RGS4 is required for dopaminergic control of striatal LTD and susceptibility to parkinsonian motor deficits. Neuron 73: 347-359.
25. Kapust R B, Tozser J, Fox J D, Anderson D E, Chemy S, et al. (2001) Tobacco etch virus protease: mechanism of autolysis and rational design of stable mutants with wild-type catalytic proficiency. Protein Eng 14: 993-1000.
26. Shankaranarayanan A, Thal D M, Tesmer V M, Roman D L, Neubig R R, et al. (2008) Assembly of high order G alpha q-effector complexes with RGS proteins. J Biol Chem 283: 34923-34934.
27. Soundararajan M, Willard F S, Kimple A J, Turnbull A P, Ball L J, et al. (2008) Structural diversity in the RGS domain and its interaction with heterotrimeric G protein alpha-subunits. Proceedings of the National Academy of Sciences of the United States of America 105: 6457-6462.
28. Sternweis P C, Robishaw J D (1984) Isolation of two proteins with high affinity for guanine nucleotides from membranes of bovine brain. J Biol Chem 259: 13806-13813.
29. Blazer L L, Zhang H, Casey E M, Husbands S M, Neubig R R (2011) A nanomolar-potency small molecule inhibitor of regulator of G-protein signaling proteins. Biochemistry 50: 3181-3192.
30. Weisman J L, Liou A A, Shelat A A, Cohen F E, Guy R K, et al. (2006) Searching for new antimalarial therapeutics amongst known drugs. Chemical biology & drug design 67: 409-416.
31. Berman D M, Kozasa T, Gilman A G (1996) The GTPase-activating protein RGS4 stabilizes the transition state for nucleotide hydrolysis. The Journal of biological chemistry 271: 27209-27212.
32. Tesmer J J, Berman D M, Gilman A G, Sprang S R (1997) Structure of RGS4 bound to AlF4-activated G(i alpha1): stabilization of the transition state for GTP hydrolysis. Cell 89: 251-261.
33. Mao H, Zhao Q, Daigle M, Ghahremani M H, Chidiac P, et al. (2004) RGS17/RGS Z2, a novel regulator of Gi/o, Gz, and Gq signaling. The Journal of biological chemistry 279: 26314-26322.
34. Conklin B R, Chabre 0, Wong Y H, Federman A D, Bourne H R (1992) Recombinant Gq alpha. Mutational activation and coupling to receptors and phospholipase C. The Journal of biological chemistry 267: 31-34.
35. Wong Y H, Federman A, Pace A M, Zachary I, Evans T, et al. (1991) Mutant alpha subunits of Gi2 inhibit cyclic AMP accumulation. Nature 351: 63-65.
36. Krumins A M, Gilman A G (2002) Assay of RGS protein activity in vitro using purified components. Methods in enzymology 344: 673-685.
37. Marin E P, Krishna A G, Sakmar T P (2001) Rapid activation of transducin by mutations distant from the nucleotide-binding site: evidence for a mechanistic model of receptor-catalyzed nucleotide exchange by G proteins. The Journal of biological chemistry 276: 27400-27405.
38. Dai Y, Desano J, Tang W, Meng X, Meng Y, et al. (2010) Natural proteasome inhibitor celastrol suppresses androgen-independent prostate cancer progression by modulating apoptotic proteins and NF-kappaB. PLoS One 5: e14153.

We claim:

1. A method of treating or preventing a disorder mediated by aberrant G protein signaling comprising administering to a patient in need thereof a therapeutically effective amount of a compound selected from the group consisting of formula UI-5, UI-1590, UI-1907 and UI-2034

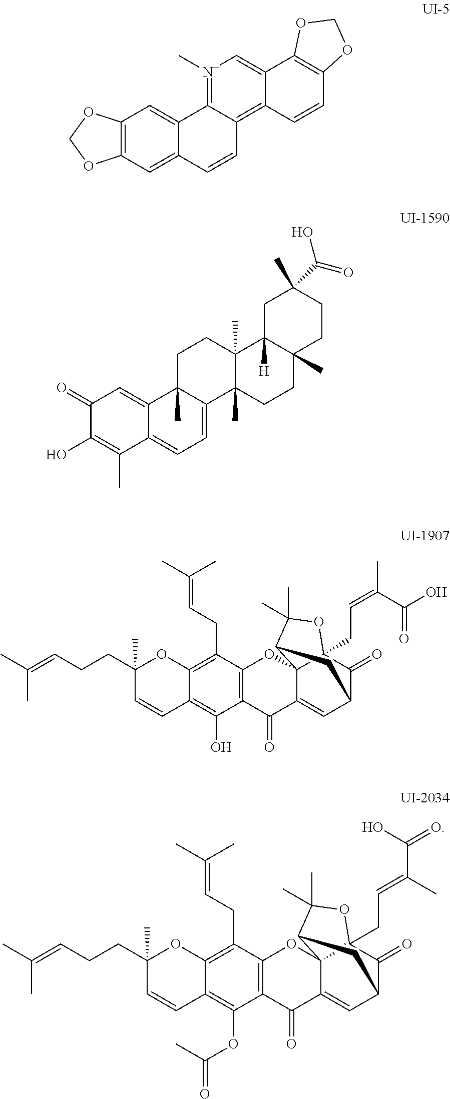

2. The method of claim 1, wherein said compound is UI-5.

3. The method of claim 1, wherein said compound is UI-1590.

4. The method of claim 1, wherein said compound is UI-1907.

5. The method of claim 1, wherein said compound is UI-2034.

6. A method of treating or preventing a Parkinson's disease comprising administering to a patient in need thereof a therapeutically effective amount of a compound selected from the group consisting of formula UI-5, UI-1590, UI-1907 and UI-2034

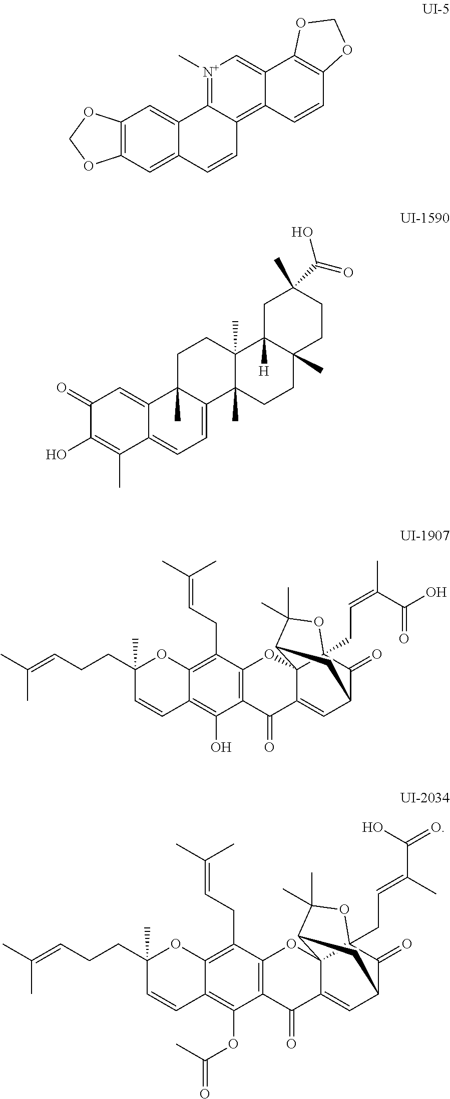

7. The method of claim 6, wherein said compound is UI-5.

8. The method of claim 6, wherein said compound is UI-1590.

9. The method of claim 6, wherein said compound is UI-1907.

10. The method of claim 6, wherein said compound is UI-2034.
